(19) United States
(12) Patent Application Publication
(10) Pub. No.: US 2018/0008378 A1
(43) Pub. Date: Jan. 11, 2018

(54) SMART ORTHODONTIC BRACKET

(71) Applicants: SREEVATSAN RAGHAVAN, NOIDA (IN); PRAFUL JAIN, Ghaziabad (IN)

(72) Inventors: SREEVATSAN RAGHAVAN, NOIDA (IN); PRAFUL JAIN, Ghaziabad (IN)

(21) Appl. No.: 15/544,674

(22) PCT Filed: Jan. 20, 2016
(86) PCT No.: PCT/IB2016/050272

§ 371 Date: Jul. 19, 2017

(30) Foreign Application Priority Data
Jan. 20, 2015 (IN) 174/DEL/2015

(51) Int. Cl. A61C 7/14; A61C 7/00; A61B 5/1455; A61C 19/04; G01N 21/88; G01L 1/16

(52) U.S. Cl. CPC A61C 7/146; G01N 21/88; G01L 1/16; A61B 5/14552; A61C 19/04; A61C 7/002; A61C 2204/005

(57) ABSTRACT

The present invention provides an improved orthodontic bracket with integrated piezosensor chip determining even the slightest change in initial installation setup of orthodontic bracket system and further notifying the subscriber electronically comprising of orthodontic bracket, for holding the tooth; at least one wire, for connecting the orthodontic brackets; and at least one sensor chip placed on the bracket and/or on the wire, wherein, at least one pulse oximeter is installed at the periphery of the bracket base to ensure force applied are within biological limits; the invention provides a detachable wireless frequency transmitter to be placed on the bracket of tie-wing to permit clinical recycling of the bracket; and optionally allowing a handheld laser scanner/photospectrometer to measure the frictional coefficient between the archwire and bracket to determine exact amount of force required for tooth retraction; to determine the bonding technique for detecting voids in adhesive; and to determine the distance between forces applied and center of mass of the tooth.

### FIELD OF THE INVENTION

The present invention relates to an improved orthodontic bracket. More particularly, the invention provides an orthodontic bracket with integrated 3D piezosensor chip and a detachable wireless frequency transmitter to bracket base to ensure force applied are within biological limits, and a method of determining force applied within biological limits.

### BACKGROUND OF THE INVENTION

Teeth are attached to the bone with little mini rubber band type fibers known as periodontal ligaments. These ligaments allow for very small natural movements of the teeth while eating or using the teeth. This ligament provides a little space between the tooth and the bone known as the periodontal ligament space. Now, suppose a pressure is applied on the tooth, pushing it in one direction. This pressure either stretches (tension) the ligaments on one side of the tooth, or squishes (compresses) the ligaments on the other side of the tooth. Therefore the tooth experiences a tension side and a compression sides. When the body senses these prolonged forces, it starts adding bone, with cells called osteoblasts on the tension side of the body. On the compression side, the body starts eating away bone with cells called osteoclasts. This system of removing bone on one side, while adding bone on the other side, is what allows teeth to move through bone. If the force is too large the system will not be very efficient. A light, continuous force is the most efficient way to move teeth. Therefore, when we put on braces, we are setting up a mechanical system to deliver forces to the teeth.

Orthodontics is a specific field of dentistry that diagnoses, prevents and treats irregularities of the teeth and face. Orthodontic treatment regarding irregularities of the teeth involves straightening of teeth, correcting an irregularity in bite, closing of unsightly gaps and bringing teeth and lips into proper alignment.

Majorly there are two types of braces removable and permanent. Removable braces can be put on or removed by patients and is custom made to fit the shape of each patient's mouth. Permanent braces such as metallic braces are the traditional braces with a system of very visible brackets and arch wires that requires specialized help for installation and removal. These metallic braces are cheap, offer more control over the desired tooth movements and therefore more popular. Although, the type of braces may vary but the goal of the treatment is the same i.e. to gradually move the teeth into the desired position.

With the exception of Invisalign brace, all other types of braces employ brackets on each and every tooth and connect them with a wire to rectify the irregularities. A dentist shall repeat the process many times over the period to set and align. A patient shall have to visit a dentist many times just to maintain a constant pressure to gradually move the teeth.

One of the major drawbacks associated with use of braces for rectification, is frequent the pain. Constant adjustments are done to maintain force on a tooth so that irregularities are treated. Although, after a while when the tooth moves under applied force, the pressure slackens; so to maintain pressure constant adjustments are done. Therefore, each time a person undergoes check-up; orthodontist makes adjustments and bends, adjusts, tightens, or replaces the wire with a thicker wire to force your teeth to shift. The orthodontist may further change the ties used to hold the archwire to the brackets and make any other necessary adjustments to your braces. Each adjustment is a step toward straight teeth, but sometimes the movement of teeth can be painful.

The prior art presented here provide modern brackets to adjust the magnitude of force so that the archwire does not slacken and relay such messages to a central processor to make adjustments, timely.

In WO2007133422A3, the invention calculates, force magnitudes and/or directions may be determined objectively using orthodontic brackets having an elastomeric member which allows one portion of the bracket to be resiliently moveable relative to at least on other portion of the bracket. In a preferred embodiment, the brackets include a lower base member, an upper bracket member, and an elastomeric layer interposed between the lower base and upper bracket members. The orthodontic bracket is advantageously employed as part of a system whereby the orthodontic bracket includes an elastomeric member which allows at least one portion of the bracket to be resiliently movable relative to at least one other portion of the bracket in response to an applied force, and at least one force-responsive sensor operatively associated with the orthodontic bracket for generating a detectable signal in response to movement of the at least one and other portions of the bracket. A detector is provided so as to allow for the wireless detection of the force-responsive signal generated by the force-responsive sensor and issue an output signal in response thereto. A processor receives the output signal from the detector to provide an indication of magnitude and/or direction of the force applied to the orthodontic bracket.

In EP1505921B1, the invention relates to an orthodontic bracket (B) serving to fix a tight-fitting orthodontic appliance to a tooth, comprising a bracket base (2), which is to be fixed to a tooth (1), and a bracket attachment (3) for attaching a force/pressure and/or torque application device. According to the invention, at least one sensor device is provided between the bracket base (2) and the bracket attachment (3) for measuring a force, which is imparted by the bracket attachment (3) to the bracket base (2), an imparted pressure and/or an imparted torque. The invention also relates to a tight-fitting orthodontic appliance comprising at least one orthodontic bracket of the aforementioned type. The sensor device (4) integrated inside bracket (B) makes it possible to measure the forces, pressures and/or torques that are actually acting on the tooth (1) thereby enabling the orthodontist to take this data into account during the use of the orthodontic appliance. The invention also relates to a removable orthodontic appliance.

However, these closest prior art provide modern brackets to adjust the magnitude of force so that the archwire does not slacken but there are no prior arts that accounts for the pain and hence adjust the archwire automatically via an external controller.

Due to lack in technology the amount of pain suffered by each patient's is different as the bone physiology is unique and responds differently to the same stress of the orthodontic appliance. Most of the time a patient's ability to withstand pain is the only parameter for such adjustments. Accordingly, there is a need for a solution to overcome the shortcomings in the state of the art. Therefore, there is requirement for the brackets apply force within the biological limits for Internal pain due to the pain caused by changes in blood flow that occur when the braces apply pressure to the teeth.

Further, the entire prior art installs their RFID tags on the bracket base, which is removed with a blow torch that damages the wireless transmitter, hence they are not recyclable.

The present invention removes all the drawbacks of the prior art.

### OBJECT OF THE INVENTION

Accordingly, the main object of the invention is to provide a smart orthodontic bracket system fitted with at least one sensor chip such as a 3D piezosensor chip, which telemetrically provides information about the change from the initial setup of the orthodontic bracket system wherein additional pulse oximeter at the periphery of the bracket base to ensure force levels within biological limits.

Another main object of the invention is to provide a method for automatically determining even the slightest change in initial installation setup of orthodontic bracket system such as lose wire(s), broken bracket(s), conformational or spatial change, change in pressure, strain or force etc.; and notifying the dentist and/or user electronically about the change and set up a follow up or adjustment visits automatically.

Yet another object of the invention is configure a handheld laser scanner/photospectrometer to measure the frictional coefficient between the archwire and bracket to determine exact amount of force required for tooth retraction, or if wire is worn out-advocate change of wire.

Yet another object of the invention is to configure a handheld laser scanner/photospectrometer to determine the bonding technique for detecting voids in adhesive.

Yet another object of the invention is to configure a handheld laser scanner/photospectrometer to determine the distance between forces applied and center of mass of the tooth as with age and recession of the gums the center of mass will change.

Yet another object of the invention is to configure a detachable wireless frequency transmitter to be placed on the bracket of tie-wing to permit clinical recycling of the bracket, which is conventionally done by heating the bracket base with a blow torch that damages the wireless transmitter but the present invention provides piezo-sensors on the bracket of tie-wing that shall allow piezo-sensors to withstand the temperature.

Yet another object of the invention is to connect the orthodontic bracket telemetrically to a server for data storage.

Still another object of the invention is to make this orthodontic bracket compatible with other remotely/hand held devices.

Yet another object of the invention is to accurately determine the amount of pressure being exerted on each tooth to maintain the level of safety.

### SUMMARY OF THE INVENTION

Accordingly, the present invention relates to an improved orthodontic bracket. The invention provides an orthodontic bracket with integrated 3D piezosensor chip and a detachable wireless frequency transmitter to ensure force levels exerted on each tooth are within biological limits and a method of determining even the slightest change in initial installation setup of orthodontic bracket system; and further notifying the subscriber electronically.

The orthodontic bracket comprises plurality of orthodontic brackets, for holding the tooth; at least one wire, for connecting the orthodontic brackets; and at least one sensor chip placed on the bracket and/or on the wire, wherein the sensor chip gathers and relays information related to change in installation set up of the orthodontic bracket system telemetrically and provides information about the change from the initial setup of the orthodontic bracket system and allows the clinician to determine if a wire becomes passive and needs to be changed, if the bracket has been de-bonded or requires replacement etc. No elastomeric components are installed due to problem of force decay therefore installing the piezosensor at the base of the bracket.

In an embodiment of the current invention, the invention provides a smart orthodontic bracket system fitted with at least one sensor chip such as a 3D piezosensor chip and a detachable wireless frequency transmitter, which ensure force levels exerted on each tooth are within biological limits and, telemetrically and relays information about the change from the initial setup of the orthodontic bracket system wherein additional pulse oximeter at the periphery of the bracket base is to ensure force levels are within biological limits.

In an embodiment of the current invention, the invention allows configuration of a handheld laser scanner/photospectrometer to measure the frictional coefficient between the archwire and bracket to determine exact amount of force required for tooth retraction, or if wire is worn out then advocate change of wire; to determine the bonding technique for detecting voids in adhesive; and to determine the distance between forces applied and center of mass of the tooth, as with age and recession of the gums the center of mass changes.

In an embodiment of the current invention, the invention provides a detachable wireless frequency transmitter to be placed anywhere on the bracket of tie-wing to permit clinical recycling of the bracket, as usually the bracket base is heated with a blow torch to remove the wireless transmitter and then autoclaved, but the present invention provides detachable wireless frequency transmitter (RFID) on the bracket of tie-wing.

In an embodiment of the current invention, the invention provides a method of determining force applied within biological limits comprising steps of a. installing atleast one pulse oximeter at the periphery of the bracket base to control the amount of force applied; b. installing atleast one detachable wireless frequency transmitter to be placed on the bracket of tie-wing to communicate with external and internal devices and sensors; c. utilizing atleast one handheld scanner to generate data and, collate and analyse data from steps a and b; and d. transmit the data from step c to a central processor for measuring the frictional coefficient between the archwire and bracket, determining the exact amount of force required for tooth retraction, determining the bonding technique for detecting voids in adhesive and determining the distance between forces applied and center of mass of the tooth; and notifying a registered user of the processed data from step d. The pulse oximeter is a programmable device to control the amount of force applied. The central processor is attached to a Visual display unit, a communication unit and a input device. The handheld scanner is preferably a laser scanner/photospectrometer.

### BRIEF DESCRIPTION OF DRAWINGS

A complete understanding of the device and method of the present invention may be obtained by reference to the following drawing:

FIG. 1, shows smart orthodontic bracket according to the present invention;

FIG. 2, the smart orthodontic bracket showing the top view of the base is shown;

FIG. 3, the smart orthodontic bracket showing the top view of the bracket;

FIG. 4, the smart orthodontic bracket showing the top view of the bracket; and

FIG. 5, shows a handheld laser scanner/photospectrometer to measure the frictional coefficient between the archwire and bracket.

### DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

In an embodiment of the present invention, the invention provides for a smart orthodontic bracket system fitted with at least one 3D piezosensor chip and an RFID chip, which controls the amount of pressure exerted on a tooth and telemetrically relays information about the slightest change in the installation of the orthodontic bracket fitted.

In another preferred embodiment of the present invention, the smart orthodontic bracket system comprises of plurality of orthodontic brackets, for holding the tooth; at least one wire, for connecting the orthodontic brackets; and at least one sensor chip placed on the bracket and/or on the wire, wherein the sensor chip gathers and relays information related to change in installation set up of the orthodontic bracket system telemetrically and provides information about the change from the initial setup of the orthodontic bracket system and allows the clinician to determine if a wire becomes passive and needs to be changed, if the bracket has been de-bonded or requires replacement etc. The orthodontic brackets and wires are preferably made up of metal such as steel, titanium etc. The sensor chip is most preferably a 3D piezosensor chip designed to measure changes in pressure, strain or force, however other sensor chips may also be installed either alone or in combination. The installation information shall include but not limited to the tension on the wires, pressure exerted on the individual tooth, alignment of the tooth such as crowding or spacing, any other conformational or spatial change, change in the pressure, strain or force etc.

In yet another embodiment of the present invention, the invention provides a method of automatically notifying change in the installation set up of orthodontic bracket system to a subscriber. The method comprises of collecting the installation information when the smart orthodontic bracket system is installed and setting the same as reference; storing the information on a remote server and other memory based hand held device(s); collecting the installation information in fixed or variable programmed or non programmed interval(s); comparing the initial data and the latest data; and finally notifying the receiver of the percent change from the initial installation pressure, strain or force kept as reference.

In an embodiment of the current invention, the invention provides a smart orthodontic bracket system fitted with at least one sensor chip such as a 3D piezosensor chip, which telemetrically provides information about the change from the initial setup of the orthodontic bracket system wherein additional pulse oximeter at the periphery of the bracket base is to ensure force levels are within biological limits.

In an embodiment of the current invention, the invention allows configuration of a handheld laser scanner/photospectrometer to measure the frictional coefficient between the archwire and bracket to determine exact amount of force required for tooth retraction, or if wire is worn out- advocate change of wire; to determine the bonding technique for detecting voids in adhesive; and to determine the distance between forces applied and center of mass of the tooth, as with age and recession of the gums the center of mass changes.

In an embodiment of the current invention, the invention provides a detachable wireless frequency transmitter to be placed on the bracket of tie-wing to permit clinical recycling of the bracket, which is conventionally done by heating the bracket base with a blow torch that damages the wireless transmitter but the present invention provides piezosensors on the bracket of tie-wing that shall allow piezosensors to withstand the temperature.

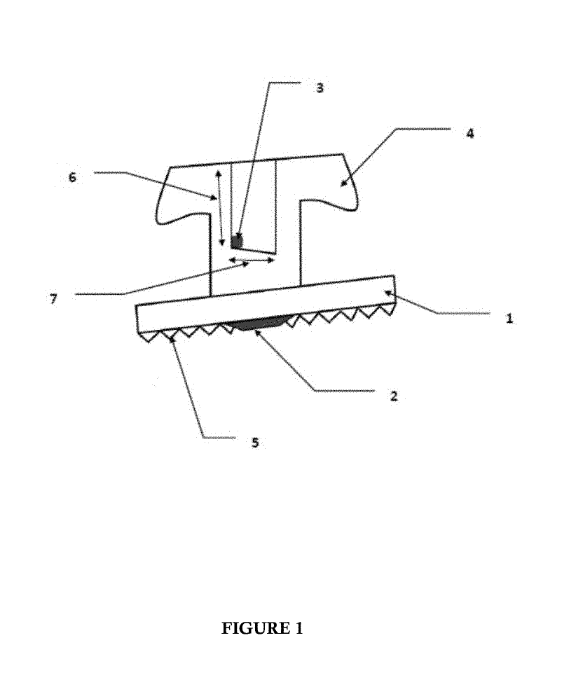

As shown in FIG. 1, the smart orthodontic bracket 10 has tie wings 4, grooves 5 for bonding, a 3D piezosensor 2 positioned at a particular slot height 6 in a manner so as to sense wire interaction. Another sensor (not shown) is located at the base 1 of the bracket which detects defending. The 3D piezosensor 2 and RFID chip (not shown) for a smart orthodontic bracket system fitted which controls the amount of pressure exerted on a tooth and telemetrically relays information about the slightest change in the installation of the orthodontic bracket fitted.

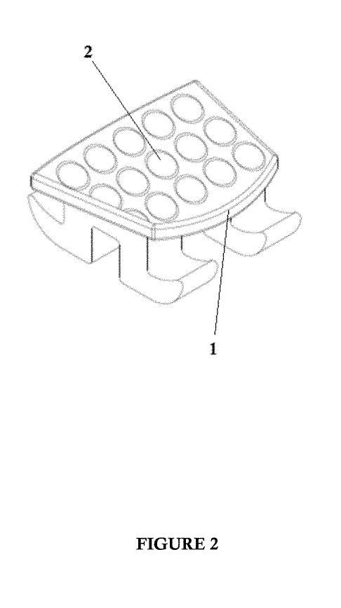

As shown in FIG. 2, the smart orthodontic bracket showing the top view of the base is shown. The base 1 is shown to have specially designed multiple grooves 5 to accommodate 3D piezosensors (not shown in the figure). The base 1 is attached to the teeth. The base 1 rests on the teeth due to traction provided by multiple grooves 5.

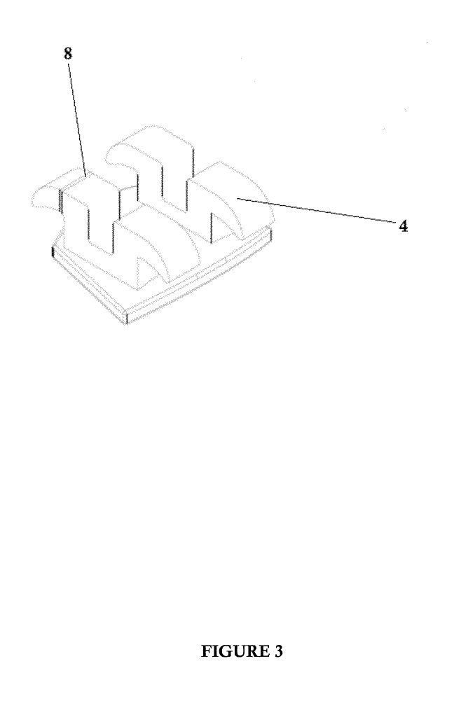

As shown in FIG. 3, the smart orthodontic bracket showing the top view of the bracket. The view elucidates the detachable wireless frequency transmitter 8 such as RFID installed on the tie wings.

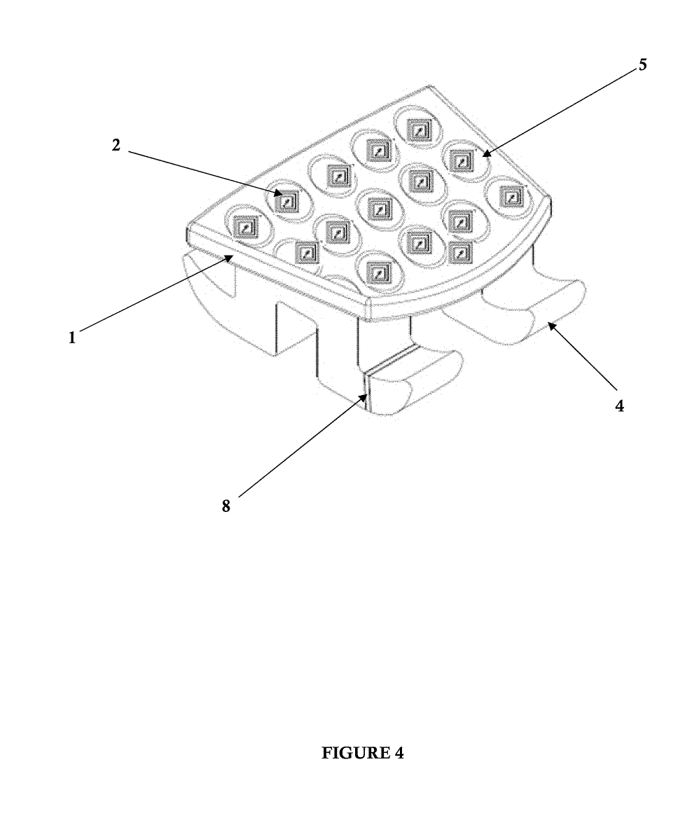

As shown in FIG. 4, the smart orthodontic bracket showing the top view of the bracket, having installed grooves 5 to accommodate 3D piezosensors 2. The smart orthodontic bracket showing the detachable wireless frequency transmitter 8 such as RFID installed on the tie wings 4. The 3D piezosensors 2 installed in multiple grooves 5 to ensure that the force applied are within biological limits. Further, the detachable arrangement of detachable wireless frequency transmitter 8 is specifically highlighted.

In an embodiment of the current invention, the invention provides a method of determining force applied within biological limits comprising steps of a. installing atleast one pulse oximeter at the periphery of the bracket base to control the amount of force applied; b. installing atleast one detachable wireless frequency transmitter to be placed on the bracket of tie-wing to communicate with external and internal devices and sensors; c. utilizing atleast one handheld scanner to generate data and, collate and analyse data from steps a and b; and d. transmit the data from step c to a central processor for measuring the frictional coefficient between the archwire and bracket, determining the exact amount of force required for tooth retraction, determining the bonding technique for detecting voids in adhesive and determining the distance between forces applied and center of mass of the tooth; and notifying a registered user of the processed data from step d. The pulse oximeter is a programmable device to control the amount of force applied. The central processor is attached to a Visual display unit, a communication unit and a input device. The handheld scanner is preferably a laser scanner/photospectrometer.

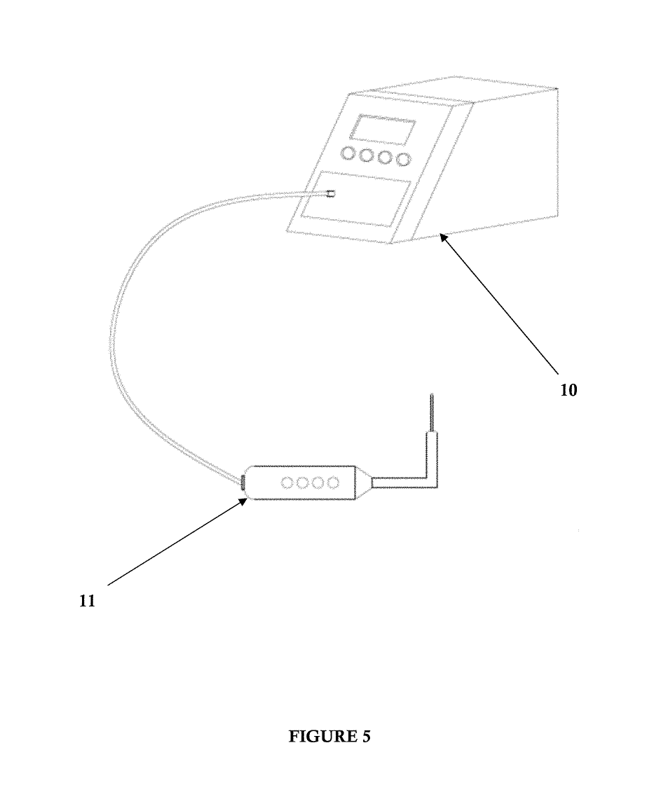

As shown in FIG. 5, a handheld laser scanner/photospectrometer to measure the frictional coefficient between the archwire and bracket. The scanner/photospectrometer 10 shown here, augmented with a probe 11 to determine the exact amount of force required for tooth retraction and determines if the wire is worn out; to determine the bonding technique for detecting voids in adhesive; and to determine the distance between forces applied and center of mass of the tooth, as with age and recession of the gums the center of mass changes.

Thus the present technology undermines the drawbacks of state of the art and provides an orthodontic bracket system fitted with at least one piezosensor chip, which telemetrically provides information about the change from the initial setup of the orthodontic bracket system and allows the clinician to determine if a wire becomes passive and needs to be changed, if the bracket has been de-bonded and requires replacement etc. All the data collected shall be transmitted to a local server that shall be synchronized with the dentist's database that would update information for visits.

We claim:

1. An improved orthodontic bracket comprising
an orthodontic bracket, for holding the tooth; at least one wire, for connecting the orthodontic brackets; and at least one sensor chip placed on the bracket and/or on the wire, wherein:
atleast one pulse oximeter is installed at the periphery of the bracket base to ensure force applied within the biological limits;
a detachable wireless frequency transmitter to be placed on the bracket of a tie-wing to permit clinical recycling of the bracket; and
optionally allowing a handheld laser scanner/photospectrometer to measure the frictional coefficient between an archwire and the bracket to determine exact amount of force required for tooth retraction.

2. The improved orthodontic bracket as claimed in claim 1, wherein said handheld laser scanner/photospectrometer is used to determine worn out wire.

3. The improved orthodontic bracket as claimed in claim 1, wherein said piezosensors on the bracket of the tie-wing allows piezosensors to withstand the temperature during clinical recycling.

4. The improved orthodontic bracket as claimed in claim 1, wherein the sensor chip gathers and relays information related to change in installation set up of the orthodontic bracket system telemetrically and provides information about the change from the initial setup of the orthodontic bracket system and allows the clinician to determine if a wire becomes passive and needs to be changed, if the bracket has been de-bonded or requires replacement.

5. The improved orthodontic bracket as claimed in claim 1, wherein the two bracket members are not connected by an elastomeric component therefore there is force decay of the elastomeric component in saliva causing improper force level detection.

6. The improved orthodontic bracket as claimed in claim 1, wherein 3D piezosensor detects frictional force and also measure the integrity of the bonding of the bracket to the teeth.

7. A method of determining force applied within biological limits comprising steps of:
a) installing atleast one pulse oximeter at the periphery of the bracket base to control the amount of force applied;
b) installing atleast one detachable wireless frequency transmitter to be placed on the bracket of tie-wing to communicate with external and internal devices and sensors;
c) utilizing atleast one handheld scanner to generate data and, collate and analyse data from steps a) and b);
d) transmit the data from step c) to the central processor for measuring the frictional coefficient between the archwire and bracket, determining the exact amount of force required for tooth retraction, determining the bonding technique for detecting voids in adhesive and determining the distance between forces applied and center of mass of the tooth; and
e) notifying a registered user of the processed data from step d).

8. The method as claimed in claim 7, wherein said pulse oximeter is a programmable device to control the amount of force applied.

9. The method as claimed in claim 7, wherein said central processor is attached to a Visual display unit, a communication unit and an input device.

10. The method as claimed in claim 7, wherein the handheld scanner is preferably the laser scanner/photospectrometer.
